(19) United States
(12) Patent Application Publication
(10) Pub. No.: US 2008/0095768 A1
(43) Pub. Date: Apr. 24, 2008

(54) USE OF ALLOGENEIC EFFECTOR CELLS AND ANTI-CS1 ANTIBODIES FOR SELECTIVE KILLING OF MULTIPLE MYELOMA CELLS

(75) Inventors: Daniel AFAR, Fremont, CA (US); Frits VAN RHEE, Little Rock, AR (US)

Correspondence Address: PDL BIOPHARMA, INC.;Attn: Legal Department, 1400 Seaport Boulevard, Redwood City, CA, 94063, US

(73) Assignees: PDL BIOPHARMA, INC., Fremont, CA (US); UNIVERSITY OF ARKANSAS FOR MEDICAL SCIENCE, Little Rock, AR (US)

(21) Appl. No.: 11/835,260

(22) Filed: Aug. 7, 2007

Related U.S. Application Data
(60) Provisional application No. 60/836,353, filed on Aug. 7, 2006.

(51) Int. Cl. A61K 39/395; A61K 31/19; A61K 31/56; A61P 43/00; A61K 31/675; A61K 35/00

(52) U.S. Cl. 424/133.1; 424/93.71; 514/171; 514/570; 514/81

(57) ABSTRACT

Methods for treating MM using anti-CS1 antibodies are provided herein.

### 1. CROSS REFERENCE TO RELATED APPLICATIONS

This application claims benefit under 35 U.S.C. § 119(e) to application Ser. No. 60/836,353, filed Aug. 7, 2006, the contents of which are incorporated herein by reference.

### 2. BACKGROUND

Multiple myeloma (“MM”) represents a malignant proliferation of plasma cells derived from a single clone. The terms multiple myeloma and myeloma are used interchangeably to refer to the same condition. The myeloma tumor, its products, and the host response to it result in a number of organ dysfunctions and symptoms of bone pain or fracture, renal failure, susceptibility to infection, anemia, hypocalcemia, and occasionally clotting abnormalities, neurologic symptoms and vascular manifestations of hyperviscosity. See D. Longo, in Harrison's Principles of Internal Medicine 14th Edition, p. 713 (McGraw-Hill, New York, 1998). Human multiple myeloma remains an incurable hematological malignancy that affects 14,400 new individuals in the United States annually (See Anderson, K. et al., Introduction. Seminars in Oncology 26:1 (1999)). No effective long-term treatment currently exists for MM. It is a malignant disease of plasma cells, manifested as hyperproteinemia, anemia, renal dysfunction, bone lesions, and immunodeficiency. MM is difficult to diagnose early because there may be no symptoms in the early stage. The disease has a progressive course with a median duration of survival of six months when no treatment is given. Systemic chemotherapy is the main treatment, and the current median of survival with chemotherapy is about three years, however fewer than 5% live longer than 10 years (See Anderson, K. et al., Annual Meeting Report 1999. Recent Advances in the Biology and Treatment of Multiple Myeloma (1999)).

Additional treatment strategies include high-dose therapy with autologous hematopoietic cell transplantation (HCT), tandem autografts, and high-dose conditioning with allogeneic HCT. Allogeneic HCT is associated with a higher frequency of sustained remissions and a lower risk of relapse due to the graft-versus-tumor activity through immune response against minor antigen differences between donor and host. Unfortunately, allogeneic HCT is also associated with high transplantation related mortality, due in part to graft versus host disease (GVHD). Approaches using nonmyeloablative conditioning and novel posttransplantation immunosuppression to assure engraftment and graft-versus-tumor effects have reduced the transplantation related mortality (see, e.g., Maloney, et al., 2003, Blood, 102:3447-3454). However, new methods of treatment are needed to further reduce transplantation related mortality and extend the duration of remission in treated patients.

### 3. SUMMARY

Described herein are compositions and methods useful for exploiting the anti-tumor properties of anti-CS1 antibodies and the graft-versus-tumor properties of allogeneic effector cells, in particular, alloreactive natural killer (NK) cells.

The anti-CS1 antibodies described herein are recombinant monoclonal antibodies directed to human CS1. CS1 (CD2-subset1) is also known as SLAMF7, CRACC, 19A, APEX-1, and FOAP12 (Genbank Accession Number NM_{—}021181.3). CS1, is a glycoprotein that is highly expressed in bone marrow samples from patients diagnosed with MM. In both in vitro and in vivo studies, anti-CS1 antibodies, such as HuLuc63, exhibit significant anti-myeloma activity (see, e.g., U.S. Patent Publication Nos. 2005/0025763 and 2006/0024296, the contents of which are incorporated herein by reference). By way of example, but not limitation, the anti-CS1 antibody HuLuc63 effectively mediates lysis of myeloma cells via antibody dependent cellular cytotoxicity (ADCC) (see, e.g., U.S. Patent Publication Nos. 2005/0025763, the contents of which are incorporated herein by reference). In a myeloma mouse tumor model, treatment with HuLuc63 significantly reduced tumor mass by more than 50% (see, e.g., U.S. Patent Publication Nos. 2005/0025763, the contents of which are incorporated herein by reference).

NK cells have antigen-independent tumor cytotoxicity and have been shown in murine models to control and prevent tumor growth and dissemination (Moretta, et al., 2002, Nat. Immunol. 3:6-8). Alloreactive, allogeneic NK cells mismatched for killer immunoglobulin-like receptors (KIRs) are more cytotoxic to tumor targets, i.e., renal cell carcinoma and melanoma, than allogeneic NK cells matched for KIRs (Igarashi et al., 2004, Blood, 104:170-177). One advantage associated with the use of alloreactive NK cells over other allogeneic effector cells is that alloreactive NK cells do not induce a graft-versus-host reaction (Ruggeri, et al., 2002, Science, 295:2097-2100).

The present disclosure relates to compositions and methods for treating a spectrum of MM patients, including asymptomatic and symptomatic. In particular, the methods relate to the administration of allogeneic effector cells in combination with anti-CS1 antibodies. Anti-CS1 antibodies are typically administered as an intravenous infusion at doses ranging from 0.5 to 20 mg/kg once every week to once a month. Other therapeutic agents, such as targeted agents, conventional chemotherapy agents, hormonal therapy agents, and supportive care agents can be used as deemed necessary by the clinician or practitioner administering the therapy.

In some embodiments, administration of the pharmaceutical compositions described herein elicits at least one of the beneficial responses as defined by the European Group for Blood and Marrow transplantation (EBMT). For example, administration of the pharmaceutical compositions described herein can result in a complete response, partial response, minimal response, no change, or plateau.

### 4. BRIEF DESCRIPTION OF THE FIGURES

FIG. 1 depicts CS1 mRNA expression in CD138+ plasma cells; and

FIG. 2 depicts enhanced lysis of myeloma cells by allogeneic NK cells following pretreatment with HuLuc63.

### 5. DETAILED DESCRIPTION

The methods described herein combine the administration of allogeneic effectors cells with anti-CS1 antibodies to potentiate or complement the anti-myeloma activities of the other. Typically, the methods can be used to treat patients diagnosed with asymptomatic MM, and symptomatic MM, ranging from newly diagnosed to late stage relapsed/refractory.

Examples of suitable anti-CS1 antibodies for use in the methods described herein include, but are not limited to, isolated antibodies that bind one or more of the three epitope clusters identified on CS1 and monoclonal antibodies produced by the hybridoma cell lines: Luc2, Luc3, Luc15, Luc22, Luc23, Luc29, Luc32, Luc34, Luc35, Luc37, Luc38, Luc39, Luc56, Luc60, Luc63, Luc69, LucX.1, LucX.2 or Luc90. These monoclonal antibodies are named as the antibodies: Luc2, Luc3, Luc15, Luc22, Luc23, Luc29, Luc32, Luc34, Luc35, Luc37, Luc38, Luc39, Luc56, Luc60, Luc63, Luc69, LucX and Luc90, respectively, hereafter. Humanized versions are denoted by the prefix “hu” (see, e.g., U.S. Patent Publication Nos. 2005/0025763 and 2006/0024296, the contents of which are incorporated herein by reference).

In some embodiments, suitable anti-CS1 antibodies include isolated antibodies that bind one or more of the three epitope clusters identified on CS1 (SEQ ID NO: 1, Table 1 below; see, e.g., U.S. Patent Publication No. 2006/0024296, the content of which is incorporated herein by reference). As disclosed in U.S. Patent Publication No. 2006/0024296 and shown below in Table 1, the CS1 antibody binding sites have been grouped into 3 epitope clusters:
(1) the epitope defined by Luc90, which binds to hu50/mu50 (SEQ ID NO: 2). This epitope covers from about amino acid residue 23 to about amino acid residue 151 of human CS1. This epitope is resided within the domain 1 (V domain) of the extracellular domain. This epitope is also recognized by Luc34, LucX (including LucX.1 and LucX.2) and Luc69.
(2) the epitope defined by Luc38, which binds to mu25/hu75 (SEQ ID NO: 3) and hu50/mu50 (SEQ ID NO: 81). This epitope likely covers from about amino acid residue 68 to about amino acid residue 151 of human CS1. This epitope is also recognized by Luc5.
(3) the epitope defined by Luc63, which binds to mu75/hu25 (SEQ ID NO: 4). This epitope covers from about amino acid residue 170 to about amino acid residue 227 of human CS1. This epitope is resided within domain 2 (C2 domain) of human CS1. This epitope is also recognized by Luc4, Luc12, Luc23, Luc29, Luc32 and Luc37.

The methods and pharmaceutical compositions are addressed in more detail below, but typically include at least one anti-CS1 antibody as described above. In some embodiments, the pharmaceutical compositions include the anti-CS1 antibody HuLuc63. HuLuc63 is a humanized recombinant monoclonal IgG1 antibody directed to human CS1. The amino acid sequence for the heavy chain variable region (SEQ ID NO: 5) and the light chain variable region (SEQ ID NO: 6) for HuLuc63 is disclosed in U.S. Pat. No. Publication No. 2005/0025763, the content of which is incorporated herein by reference, and in Table 1.

TABLE 1

| SEQ ID | |
|---|---|
| NO: | Amino Acid Sequence |
| SEQ ID | Met Ala Gly Ser Pro Thr Cys Leu Thr Leu Ile |
| NO: 1 | Tyr Ile Leu Trp Gln Leu Thr Gly Ser Ala Ala |
| | Ser Gly Pro Val Lys Glu Leu Val Gly Ser Val |
| | Gly Gly Ala Val Thr Phe Pro Leu Lys Ser Lys |
| | Val Lys Gln Val Asp Ser Ile Val Trp Thr Phe |
| | Asn Thr Thr Pro Leu Val Thr Ile Gln Pro Glu |
| | Gly Gly Thr Ile Ile Val Thr Gln Asn Arg Asn |
| | Arg Glu Arg Val Asp Phe Pro Asp Gly Gly Tyr |
| | Ser Leu Lys Leu Ser Lys Leu Lys Lys Asn Asp |
| | Ser Gly Ile Tyr Tyr Val Gly Ile Tyr Ser Ser |
| | Ser Leu Gln Gln Pro Ser Thr Gln Glu Tyr Val |
| | Leu His Val Tyr Glu His Leu Ser Lys Pro Lys |
| | Val Thr Met Gly Leu Gln Ser Asn Lys Asn Gly |
| | Thr Cys Val Thr Asn Leu Thr Cys Cys Met Glu |
| | His Gly Glu Glu Asp Val Ile Tyr Thr Trp Lys |
| | Ala Leu Gly Gln Ala Ala Asn Glu Ser His Asn |
| | Gly Ser Ile Leu Pro Ile Ser Trp Arg Trp Gly |
| | Glu Ser Asp Met Thr Phe Ile Cys Val Ala Arg |
| | Asn Pro Val Ser Arg Asn Phe Ser Ser Pro Ile |
| | Leu Ala Arg Lys Leu Cys Glu Gly Ala Ala Asp |
| | Asp Pro Asp Ser Ser Met Val Leu Leu Cys Leu |
| | Leu Leu Val Pro Leu Leu Leu Ser Leu Phe Val |
| | Leu Gly Leu Phe Leu Trp Phe Leu Lys Arg Glu |
| | Arg Gln Glu Glu Tyr Ile Glu Glu Lys Lys Arg |
| | Val Asp Ile Cys Arg Glu Thr Pro Asn Ile Cys |
| | Pro His Ser Gly Glu Asn Thr Glu Tyr Asp Thr |
| | Ile Pro His Thr Asn Arg Thr Ile Leu Lys Glu |
| | Asp Pro Ala Asn Thr Val Tyr Ser Thr Val Glu |
| | Ile Pro Lys Lys Met Glu Asn Pro His Ser Leu |
| | Leu Thr Met Pro Asp Thr Pro Arg Leu Phe Ala |
| | Tyr Glu Asn Val Ile |
| SEQ ID | Met Ala Gly Ser Pro Thr Cys Leu Thr Leu Ile |
| NO: 2 | Tyr Ile Leu Trp Gln Leu Thr Gly Ser Ala Ala |
| | Ser Gly Pro Val Lys Glu Leu Val Gly Ser Val |
| | Gly Gly Ala Val Thr Phe Pro Leu Lys Ser Lys |
| | Val Lys Gln Val Asp Ser Ile Val Trp Thr Phe |
| | Asn Thr Thr Pro Leu Val Thr Ile Gln Pro Glu |
| | Gly Gly Thr Ile Ile Val Thr Gln Asn Arg Asn |
| | Arg Glu Arg Val Asp Phe Pro Asp Gly Gly Tyr |
| | Ser Leu Lys Leu Ser Lys Leu Lys Lys Asn Asp |
| | Ser Gly Ile Tyr Tyr Val Gly Ile Tyr Ser Ser |
| | Ser Leu Gln Gln Pro Ser Thr Gln Glu Tyr Val |
| | Leu His Val Tyr Glu His Leu Ser Lys Pro Lys |
| | Val Thr Ile Asp Arg Gln Ser Asn Lys Asn Gly |
| | Thr Cys Val Ile Asn Leu Thr Cys Ser Thr Asp |
| | Gln Asp Gly Glu Asn Val Thr Tyr Ser Trp Lys |
| | Ala Val Gly Gln Gly Asp Asn Gln Phe His Asp |
| | Gly Ala Thr Leu Ser Ile Ala Trp Arg Ser Gly |
| | Glu Lys Asp Gln Ala Leu Thr Cys Met Ala Arg |
| | Asn Pro Val Ser Asn Ser Phe Ser Thr Pro Val |
| | Phe Pro Gln Lys Leu Cys Glu Asp Ala Ala Thr |
| | Asp Leu Thr Ser Leu Arg Gly |
| SEQ ID | Met Ala Arg Phe Ser Thr Tyr Ile Ile Phe Thr |
| NO: 3 | Ser Val Leu Cys Gln Leu Thr Val Thr Ala Ala |
| | Ser Gly Thr Leu Lys Lys Val Ala Gly Ala Leu |
| | Asp Gly Ser Val Thr Phe Thr Leu Asn Ile Thr |
| | Glu Ile Lys Val Asp Tyr Val Val Trp Thr Phe |
| | Asn Thr Phe Phe Leu Ala Met Val Lys Lys Asp |
| | Gly Gly Thr Ile Ile Val Thr Gln Asn Arg Asn |
| | Arg Glu Arg Val Asp Phe Pro Asp Gly Gly Tyr |
| | Ser Leu Lys Leu Ser Lys Leu Lys Lys Asn Asp |
| | Ser Gly Ile Tyr Tyr Val Gly Ile Tyr Ser Ser |
| | Ser Leu Gln Gln Pro Ser Thr Gln Glu Tyr Val |
| | Val Leu His Val Tyr Glu His Leu Ser Lys Pro |
| | Lys Val Thr Met Gly Leu Gln Ser Asn Lys Asn |
| | Gly Thr Cys Val Thr Asn Leu Thr Cys Cys Met |
| | Glu His Gly Glu Glu Asp Val Ile Tyr Thr Trp |
| | Lys Ala Leu Gly Gln Ala Ala Asn Glu Ser His |
| | Asn Gly Ser Ile Leu Pro Ile Ser Trp Arg Trp |
| | Gly Glu Ser Asp Met Thr Phe Ile Cys Val Ala |
| | Arg Asn Pro Val Ser Arg Asn Phe Ser Ser Pro |
| | Ile Leu Ala Arg Lys Leu Cys Glu Gly Ala Ala |
| | Asp Asp Pro Asp Ser Ser Met Val |
| SEQ ID | Met Ala Arg Phe Ser Thr Tyr Ile Ile Phe Thr |
| NO: 4 | Ser Val Leu Cys Gln Leu Thr Val Thr Ala Ala |
| | Ser Gly Thr Leu Lys Lys Val Ala Gly Ala Leu |
| | Asp Gly Ser Val Thr Phe Thr Leu Asn Ile Thr |
| | Glu Ile Lys Val Asp Tyr Val Val Trp Thr Phe |
| | Asn Thr Phe Phe Leu Ala Met Val Lys Lys Asp |
| | Gly Val Thr Ser Gln Ser Ser Asn Lys Glu Arg |
| | Ile Val Phe Pro Asp Gly Leu Tyr Ser Met Lys |
| | Leu Ser Gln Leu Lys Lys Asn Asp Ser Gly Ala |
| | Tyr Arg Ala Glu Ile Tyr Ser Thr Ser Ser Gln |
| | Ala Ser Leu Ile Gln Glu Tyr Val Leu His Val |
| | Tyr Lys His Leu Ser Arg Pro Lys Val Thr Ile |
| | Asp Arg Gln Ser Asn Lys Asn Gly Thr Cys Val |
| | Ile Asn Leu Thr Cys Ser Thr Asp Gln Asp Gly |
| | Glu Asn Val Thr Tyr Ser Trp Lys Ala Val Gly |
| | Gln Ala Ala Asn Glu Ser His Asn Gly Ser Ile |
| | Leu Pro Ile Ser Trp Arg Trp Gly Glu Ser Asp |
| | Met Thr Phe Ile Cys Val Ala Arg Asn Pro Val |
| | Ser Arg Asn Phe Ser Ser Pro Ile Leu Ala Arg |
| | Lys Leu Cys Glu Gly Ala Ala Asp Asp Pro Asp |
| | Ser Ser Met Val |
| SEQ ID | Glu Val Gln Leu Val Glu Ser Gly Gly Gly Leu |
| NO: 5 | Val Gln Pro Gly Gly Ser Leu Arg Leu Ser Cys |
| | Ala Ala Ser Gly Phe Asp Phe Ser Arg Tyr Trp |
| | Met Ser Trp Val Arg Gln Ala Pro Gly Lys Gly |
| | Leu Glu Trp Ile Gly Glu Ile Asn Pro Asp Ser |
| | Ser Thr Ile Asn Tyr Ala Pro Ser Leu Lys Asp |
| | Lys Phe Ile Ile Ser Arg Asp Asn Ala Lys Asn |
| | Ser Leu Tyr Leu Gln Met Asn Ser Leu Arg Ala |
| | Glu Asp Thr Ala Val Tyr Tyr Cys Ala Arg Pro |
| | Asp Gly Asn Tyr Trp Tyr Phe Asp Val Trp Gly |
| | Gln Gly Thr Leu Val Thr Val Ser Ser |
| SEQ ID | Asp Ile Gln Met Thr Gln Ser Pro Ser Ser Leu |
| NO: 6 | Ser Ala Ser Val Gly Asp Arg Val Thr Ile Thr |
| | Cys Lys Ala Ser Gln Asp Val Gly Ile Ala Val |
| | Ala Trp Tyr Gln Gln Lys Pro Gly Lys Val Pro |
| | Lys Leu Leu Ile Tyr Trp Ala Ser Thr Arg His |
| | Thr Gly Val Pro Asp Arg Phe Ser Gly Ser Gly |
| | Ser Gly Thr Asp Phe Thr Leu Thr Ile Ser Ser |
| | Leu Gln Pro Glu Asp Val Ala Thr Tyr Tyr Cys |
| | Gln Gln Tyr Ser Ser Tyr Pro Tyr Thr Phe Gly |
| | Gln Gly Thr Lys Val Glu Ile Lys |

In some embodiments, the methods can be used to treat a MM patient who has undergone one or more therapy regimens, including conventional chemotherapy and steroids, myeloablative autologous, allogeneic, or syngeneic stem cell transplantation, tandem autologous transplantation, and/or mini non-myeloablative allogeneic transplantation.

The allogeneic effector cells can be lymphoid or myeloid cells or a combination thereof. Lymphoid cells suitable for use as allogeneic effector cells include T cells, natural killer (NK) cells, B cells, or combinations thereof. The allogeneic effector cells can be unactivated or in vitro activated as described in U.S. Pat. No. 6,143,292, the content of which is incorporated herein by reference.

In some embodiments, the allogeneic effector cells are HLA-compatible with the patient. HLA-compatible effector cells include cells that are fully HLA-matched with the patient. Alternatively, the HLA-compatible cells should be at least haploidentical with the patient. If the HLA-compatible cells are derived from a sibling of the patient, the cells preferably are fully HLA-matched with the patient, although some mismatch may be tolerated. For example, the HLA-compatible cells from a sibling may, in some cases, be single HLA locus-mismatched. If the HLA-compatible cells are derived from an unrelated individual, the cells can be fully HLA-matched, or HLA-mismatched with the patient.

In some embodiments, the allogeneic effector cells are NK cells. The allogeneic NK cells can be killer immunoglobulin-like receptor (KIR) ligand-mismatched, i.e., alloreactive NK cells, or KIR-matched (see, e.g., Igarashi et al., 2004, Blood, 104:170-177, the content of which is incorporated herein by reference). In some embodiments, autologous (KIR) ligand-mismatched NK cells are used in the methods described herein. In other embodiments, KIR-matched NK cells are used in the methods described herein.

Infusion of the allogeneic effector cells can result in complete and permanent engraftment (i.e., 100% donor cells), or in partial and transient engraftment, provided the donor cells persist sufficiently long to permit performance of allogeneic cell therapy as described herein.

Depending on the disease status and chimeric status, in some embodiments, donor lymphocyte infusions can be used following infusion of the allogeneic effector cells to establish full chimeric engraftment in patients with no GVHD (see, e.g., Badros, et al. 2002, J Clin Oncol., 20:1295-1303, the content of which is incorporated herein by reference).

The administration of allogeneic effector cells and anti-CS1 antibodies can be combined with other treatment strategies. For example, the allogeneic effector cells and an anti-CS1 antibody can be administered prior to the initiation of a treatment regimen incorporating stem cell transplantation. By way of another example, the allogeneic effector cells and an anti-CS1 antibody can be administered following a treatment regimen incorporating stem cell transplantation. The stem cell transplantation regimen can be autologous or syngeneic, tandem autologous, “mini” allogeneic, and/or combinations thereof. Accordingly, in some embodiments, allogeneic effector cells and an anti-CS1 antibody are administered after a patient has undergone a stem cell transplantation regimen. In other embodiments, allogeneic effector cells and an anti-CS1 antibody are administered before the initiation of a stem cell transplantation regimen.

In some embodiments, an anti-CS1 antibody is administered prior to the administration of allogeneic effector cells. For example, an anti-CS1 antibody can be used in a conditioning regimen, alone, or in combination with other therapeutic agents and/or total body irradiation (see, e.g., Badros et al., J. Clin. Oncol., 20:1295-1303, and Tricot, et al., 1996, Blood, 87:1196-1198, the contents of which are incorporated herein by reference). The conditioning regimen can be myeloablative or nonmyeloablative.

In other embodiments, an anti-CS1 antibody can be used in a maintenance therapy regimen. When used in a maintenance therapy regimen, the anti-CS1 antibody can be used alone or in combination with other therapeutic agents.

In other embodiments, an anti-CS1 antibody can be used in a salvage therapy regimen. When used in a salvage therapy regimen, the anti-CS1 antibody can be used alone or in combination with other therapeutic agents.

Therapeutic agents that can be used in combination with the anti-CS1 antibodies described herein include, but are not limited to, targeted agents, conventional chemotherapy agents, hormonal therapy agents, and supportive care agents. One or more therapeutic agents from the different classes, e.g., targeted, conventional chemotherapeutic, hormonal, and supportive care, and/or subclasses can be combined in the compositions described herein. The various classes described herein can be further divided into subclasses. By way of example, targeted agents can be separated into a number of different subclasses depending on their mechanism of action. As will be apparent to those of skill in the art, the agents can have more than one mechanism of action, and thus, could be classified into one or more subclasses. For purposes of the compositions and methods described herein, the following subclasses have been identified: anti-angiogenic, inhibitors of growth factor signaling, immunomodulators, inhibitors of protein synthesis, folding and/or degradation, inhibitors of gene expression, pro-apoptotic agents, agents that inhibit signal transduction and agents with “other” mechanisms of action. Typically, the mechanism of action for agents falling into the “other” subclass is unknown or poorly characterized.

For example, in some embodiments, targeted agents, such as bevacizumab, sutinib, sorafenib, 2-methoxyestradiol or 2ME2, finasunate, PTK787, vandetanib, aflibercept, volociximab, etaracizumab (MEDI-522), cilengitide, erlotinib, cetuximab, panitumumab, gefitinib, trastuzumab, TKI258, CP-751,871, atacicept, rituximab, alemtuzumab, aldesleukine, atlizumab, tocilizumab, temsirolimus, everolimus, NPI-1387, MLNM3897, HCD122, SGN-40, HLL1, huN901-DM1, atiprimod, natalizumab, bortezomib, carfilzomib, NPI-0052, tanespimycin, saquinavir mesylate, ritonavir, nelfinavir mesylate, indinavir sulfate, belinostat, LBH589, mapatumumab, lexatumumab, AMG951, ABT-737, oblimersen, plitidepsin, SCIO-469, P276-00, enzastaurin, tipifarnib, perifosine, imatinib, dasatinib, lenalidomide, thalidomide, simvastatin, and celecoxib can be combined with an anti-CS1 antibody, such as HuLuc63 and used to treat MM patients.

By way of another example, conventional chemotherapy agents, such as alklyating agents (e.g., oxaliplatin, carboplatin, cisplatin, cyclophosphamide, melphalan, ifosfamide, uramustine, chlorambucil, carmustine, mechloethamine, thiotepa, busulfan, temozolomide, dacarbazine), anti-metabolic agents (e.g., gemcitabine, cytosine arabinoside, Ara-C, capecitabine, 5FU (5-fluorouracil), azathioprine, mercaptopurine (6-MP), 6-thioguanine, aminopterin, pemetrexed, methotrexate), plant alkaloid and terpenoids (e.g., docetaxel, paclitaxel, vincristine, vinblastin, vinorelbine, vindesine, etoposide, VP-16, teniposide, irinotecan, topotecan), anti-tumor antibiotics (e.g., dactinomycin, doxorubicin, liposomal doxorubicin, daunorubicin, daunomycin, epirubicin, mitoxantrone, adriamycin, bleomycin, plicamycin, mitomycin C, carminomycin, esperamicins), and other agents (e.g., darinaparsin) can be combined with an anti-CS1 antibody, such as HuLuc63 and used to treat MM.

By way of another example, hormonal agents such as anastrozole, letrozole, goserelin, tamoxifen, dexamethasone, prednisone, and prednisilone can be combined with an anti-CS1 antibody, such as HuLuc63 and used to treat MM.

By way of another example, supportive care agents such as pamidronate, zoledonic acid, ibandronate, gallium nitrate, denosumab, darbepotin alpha, epoetin alpha, eltrombopag, and pegfilgrastim can be combined with an anti-CS1 antibody, such as HuLuc63 and used to treat MM.

In typical embodiments, an anti-CS1 antibody such as HuLuc63 is present in a pharmaceutical composition at a concentration sufficient to permit intravenous administration at 0.5 mg/kg to 20 mg/kg. In some embodiments, the concentration of an anti-CS1 antibody suitable for use in the compositions and methods described herein includes, but is not limited to, at least about 0.5 mg/kg, at least about 0.75 mg/kg, at least about 1 mg/kg, at least about 2 mg/kg, at least about 2.5 mg/kg, at least about 3 mg/kg, at least about 4 mg/kg, at least about 5 mg/kg, at least about 6 mg/kg, at least about 7 mg/kg, at least about 8 mg/kg, at least about 9 mg/kg, at least about 10 mg/kg, at least about 11 mg/kg, at least about 12 mg/kg, at least about 13 mg/kg, at least about 14 mg/kg, at least about 15 mg/kg, at least about 16 mg/kg, at least about 17 mg/kg, at least about 18 mg/kg, at least about 19 mg/kg, and at least about 20 mg/kg.

The anti-CS1 antibodies suitable for use herein can be administered in single or multiple dose regimens. Generally, an anti-CS1 antibody is administered over a period of time from about 1 to about 24 hours, but is typically administered over a period of about 1 to 2 hours. Dosages can be repeated from about 1 to about 4 weeks or more, for a total of 4 or more doses. Typically, dosages are repeated once every week, once every other week, or once a month for a minimum of 4 doses to a maximum of 52 doses.

Determination of the effective dosage, total number of doses, and length of treatment with an anti-CS1 antibody is well within the capabilities of those skilled in the art, and can be determined using a standard dose escalation study to identify the maximum tolerated dose (MTD) (see, e.g., Richardson et al., 2002, Blood, 100(9):3063-3067, the content of which is incorporated herein by reference).

In some embodiments, one or more therapeutic agents as described above can be administered in combination with an anti-CS1 antibody. The agents can be administered concurrently, prior to, or following administration of an anti-CS1 antibody.

In some embodiments, an anti-CS1 antibody is administered prior to the administration of one or more therapeutic agents (see, supra). For example, an anti-CS1 antibody can be administered approximately 0 to 60 days prior to the administration of the therapeutic agents. In some embodiments, an anti-CS1 antibody, such as HuLuc63, is administered from about 30 minutes to about 1 hour prior to the administration of the therapeutic agents, or from about 1 hour to about 2 hours prior to the administration of the therapeutic agents, or from about 2 hours to about 4 hours prior to the administration of the therapeutic agents, or from about 4 hours to about 6 hours prior to the administration of the therapeutic agents, or from about 6 hours to about 8 hours prior to the administration of the therapeutic agents, or from about 8 hours to about 16 hours prior to the administration of the therapeutic agents, or from about 16 hours to 1 day prior to the administration of the therapeutic agents, or from about 1 to 5 days prior to the administration of the therapeutic agents, or from about 5 to 10 days prior to the administration of the therapeutic agents, or from about 10 to 15 days prior to the administration of the therapeutic agents, or from about 15 to 20 days prior to the administration of the therapeutic agents, or from about 20 to 30 days prior to the administration of the therapeutic agents, or from about 30 to 40 days prior to the administration of the therapeutic agents, and from about 40 to 50 days prior to the administration of the therapeutic agents, or from about 50 to 60 days prior to the administration of the therapeutic agents.

In some embodiments, an anti-CS1 antibody, such as HuLuc63, is administered concurrently with the administration of one or more therapeutic agents as described above.

In some embodiments, an anti-CS1 antibody is administered following the administration of one or more therapeutic agents as described above. For example, an anti-CS1 antibody, such as HuLuc63, can be administered approximately 0 to 60 days after the administration of the therapeutic agents. In some embodiments, HuLuc63 is administered from about 30 minutes to about 1 hour following the administration of the therapeutic agents, or from about 1 hour to about 2 hours following the administration of the therapeutic agents, or from about 2 hours to about 4 hours following the administration of the therapeutic agents, or from about 4 hours to about 6 hours following the administration of the therapeutic agents, or from about 6 hours to about 8 hours following the administration of the therapeutic agents, or from about 8 hours to about 16 hours following the administration of the therapeutic agents, or from about 16 hours to 1 day following the administration of the therapeutic agents, or from about 1 to 5 days following the administration of the therapeutic agents, or from about 5 to 10 days following the administration of the therapeutic agents, or from about 10 to 15 days following the administration of the therapeutic agents, or from about 15 to 20 days following the administration of the therapeutic agents, or from about 20 to 30 days following the administration of the therapeutic agents, or from about 30 to 40 days following the administration of the therapeutic agents, and from about 40 to 50 days following the administration of the therapeutic agents, or from about 50 to 60 days following the administration of the therapeutic agents.

The therapeutic agents can be administered in any manner found appropriate by a clinician and are typically provided in generally accepted efficacious dose ranges, such as those described in the Physician Desk Reference, 56th Ed. (2002), Publisher Medical Economics, New Jersey. In other embodiments, a standard dose escalation can be performed to identify the maximum tolerated dose (MTD) (see, e.g., Richardson, et al. 2002, Blood, 100(9):3063-3067, the content of which is incorporated herein by reference).

In some embodiments, doses less than the generally accepted efficacious dose of a therapeutic agent can be used. For example, in various embodiments, the composition comprises a dosage that is less than about 10% to 75% of the generally accepted efficacious dose range. In some embodiments, at least about 10% or less of the generally accepted efficacious dose range is used, at least about 15% or less, at least about 25%, at least about 30% or less, at least about 40% or less, at least about 50% or less, at least about 60% or less, at least about 75% or less and at least about 90%.

The therapeutic agents can be administered singly or sequentially, or in a cocktail with other therapeutic agents, as described below. The therapeutic agents can be administered orally, intravenously, systemically by injection intramuscularly, subcutaneously, intrathecally or intraperitoneally.

The pharmaceutical compositions can exist in a solid, semi-solid, or liquid (e.g., suspensions or aerosols) dosage form. Typically, the compositions are administered in unit dosage forms suitable for single administration of precise dosage amounts. For example, an anti-CS1 antibody can be packaged in dosages ranging from about 1 to 1000 mg. In some embodiments, an anti-CS1 antibody such as HuLuc63 is packaged in a dosage at least about 1 mg, at least about 10 mg, at least about 20 mg, at least about 50 mg, at least about 100 mg, at least about 200 mg, at least about 300 mg, at least about 400 mg, at least about 500 mg, at least about 750 mg, at least about 1000 mg.

The compositions can also include, depending on the formulation desired, pharmaceutically-acceptable, nontoxic carriers or diluents, which are defined as vehicles commonly used to formulate pharmaceutical compositions for animal or human administration. The diluent is selected so as not to affect the biological activity of the combination. Examples of such diluents are distilled water, physiological saline, Ringer's solution, dextrose solution, and Hank's solution.

In addition, the pharmaceutical composition or formulation can also include other carriers, adjuvants, or nontoxic, non-therapeutic, nonimmunogenic stabilizers and the like. Effective amounts of such diluent or carrier will be those amounts that are effective to obtain a pharmaceutically acceptable formulation in terms of solubility of components, or biological activity.

Accordingly, the methods described herein can be used to develop an effective treatment strategy based on the stage of myeloma being treated (see, e.g., Multiple Myeloma Research Foundation, Multiple Myeloma: Stem Cell Transplantation 1-30 (2004); U.S. Pat. Nos. 6,143,292, and 5,928,639, Igarashi, et al. Blood 2004, 104(1): 170-177, Maloney, et al. 2003, Blood, 102(9): 3447-3454, Badros, et al. 2002, J Clin Oncol., 20:1295-1303, Tricot, et al. 1996, Blood, 87(3):1196-1198, the contents of which are incorporated herein by reference).

The staging system most widely used since 1975 has been the Durie-Salmon system, in which the clinical stage of disease (Stage I, II, or III) is based on four measurements (see, e.g., Durie and Salmon, 1975, Cancer, 36:842-854). These four measurements are: (1) levels of monoclonal (M) protein (also known as paraprotein) in the serum and/or the urine; (2) the number of lytic bone lesions; (3) hemoglobin values; and, (4) serum calcium levels. These three stages can be further divided according to renal function, classified as A (relatively normal renal function, serum creatinine value<2.0 mg/dL) and B (abnormal renal function, creatinine value≧2.0 mg/dL). A new, simpler alternative is the International Staging System (ISS) (see, e.g., Greipp et al., 2003, “Development of an international prognostic index (IPI) for myeloma: report of the international myeloma working group”, The Hematology). The ISS is based on the assessment of two blood test results, beta₂-microglobulin (β₂-M) and albumin, which separates patients into three prognostic groups irrespective of type of therapy.

Treatment of MM patients using the methods described herein typically elicits a beneficial response as defined by the European Group for Blood and Marrow transplantation (EBMT). Table 2 lists the EBMT criteria for response.

TABLE 2
EBMT/IBMTR/ABMTR¹ Criteria for Response

| | |
|---|---|
| Complete Response | No M-protein detected in serum or urine by |
| | immunofixation for a minimum of 6 weeks and |
| | fewer than 5% plasma cells in bone marrow |
| Partial Response | >50% reduction in serum M-protein level |
| | and/or 90% reduction in urine free light chain |
| | excretion or reduction to <200 mg/24 hrs for 6 |
| | weeks² |
| Minimal Response | 25-49% reduction in serum M-protein level |
| | and/or 50-89% reduction in urine free light |
| | chain excretion which still exceeds 200 mg/24 hrs |
| | for 6 weeks³ |
| No Change | Not meeting the criteria or either minimal |
| | response or progressive disease |
| Plateau | No evidence of continuing myeloma-related |
| | organ or tissue damage, <25% change in M- |
| | protein levels and light chain excretion for 3 |
| | months |
| Complete Response | No M-protein detected in serum or urine by |
| | immunofixation for a minimum of 6 weeks and |
| | fewer than 5% plasma cells in bone marrow |
| Progressive Disease | Myeloma-related organ or tissue damage |
| | continuing despite therapy or its reappearance |
| | in plateau phase, >25% increase in serum M- |
| | protein level (>5 g/L) and/or >25% increase in |
| | urine M-protein level (>200 mg/24 hrs) and/or |
| | >25% increase in bone marrow plasma cells (at |
| | least 10% in absolute terms)² |
| Relapse | Reappearance of disease in patients previously |
| | in complete response, including detection of |
| | paraprotein by immunofixation |

¹EBMT: European Group for Blood and Marrow transplantation; IBMTR: International Bone Marrow Transplant Registry; ABMTR: Autologous Blood and Marrow Transplant Registry.
²For patients with non-secretory myeloma only, reduction of plasma cells in the bone marrow by >50% of initial number (partial response) or 25-49% of initial number (minimal response) is required.
³In non-secretory myeloma, bone marrow plasma cells should increase by >25% and at least 10% in absolute terms; MRI examination may be helpful in selected patients.

Additional criteria that can be used to measure the outcome of a treatment include “near complete response” and “very good partial response”. A “near complete response” is defined as the criteria for a “complete response” (CR), but with a positive immunofixation test. A “very good partial response” is defined as a greater than 90% decrease in M protein (see, e.g., Multiple Myeloma Research Foundation, Multiple Myeloma: Treatment Overview 9 (2005)).

The response of an individual clinically manifesting at least one symptom associated with MM to the methods described herein, depends in part, on the severity of disease, e.g., Stage I, II, or III, and in part, on whether the patient is newly diagnosed or has late stage refractory MM. Thus, in some embodiments, treatment with the allogeneic effectors cells and an anti-CS1 antibody such as HuLuc63 elicits a complete response.

In other embodiments, treatment with the allogeneic effectors cells and an anti-CS1 antibody such as HuLuc63 elicits a very good partial response or a partial response.

In other embodiments, treatment with the allogeneic effectors cells and an anti-CS1 antibody such as HuLuc63 elicits a minimal response.

In other embodiments, treatment with the allogeneic effectors cells and an anti-CS1 antibody such as HuLuc63 prevents the disease from progressing, resulting in a response classified as “no change” or “plateau” by the EBMT.

Routes of administration and dosage ranges for compositions comprising an anti-CS1 antibody such as HuLuc63 and one or more therapeutic agents for treating individuals diagnosed with MM, can be determined using art-standard techniques, such as a standard dose escalation study to identify the MTD (see, e.g., Richardson, et al. 2002, Blood, 100(9):3063-3067, the content of which is incorporated herein by reference).

Typically, an anti-CS1 antibody such as HuLuc63 will be administered intravenously. Administration of the other therapeutic agents described herein can be by any means known in the art. Such means include oral, rectal, nasal, topical (including buccal and sublingual) or parenteral (including subcutaneous, intramuscular, intravenous and intradermal) administration and will depend in part, on the available dosage form. For example, therapeutic agents that are available in a pill or capsule format typically are administered orally. However, oral administration generally requires administration of a higher dose than does intravenous administration. Determination of the actual route of administration that is best in a particular case is well within the capabilities of those skilled in the art, and in part, will depend on the dose needed versus the number of times per month administration is required.

Factors affecting the selected dosage of an anti-CS1 antibody such as HuLuc63 and the therapeutic agents used in the compositions and methods described herein, include, but are not limited to, the type of agent, the age, weight, and clinical condition of the recipient patient, and the experience and judgment of the clinician or practitioner administering the therapy. Generally, the selected dosage should be sufficient to result in no change, but preferably results in at least a minimal change. An effective amount of a pharmaceutical agent is that which provides an objectively identifiable response, e.g., minimal, partial, or complete, as noted by the clinician or other qualified observer, and as defined by the EBMT.

Generally, an anti-CS1 antibody such as HuLuc63 is administered as a separate composition from the composition(s) comprising the therapeutic agents as described above. As discussed above, the therapeutic agents can each be administered as a separate composition, or combined in a cocktail and administered as a single combined composition. In some embodiments, the compositions comprising an anti-CS1 antibody such as HuLuc63 and one or more therapeutic agents are administered concurrently. In other embodiments, an anti-CS1 antibody such as HuLuc63 can be administered prior to the administration of composition(s) comprising the therapeutic agent(s). In yet other embodiments, an anti-CS1 antibody such as HuLuc63 is administered following the administration of composition(s) comprising the therapeutic agent(s).

In those embodiments in which an anti-CS1 antibody such as HuLuc63 is administered prior to or following the administration of one or more therapeutic agents as described above, determination of the duration between the administration of the anti-CS1 antibody and administration of the agents is well within the capabilities of those skilled in the art, and in part, will depend on the dose needed versus the number of times per month administration is required.

Doses of anti-CS1 antibodies used in the methods described herein typically range between 0.5 mg/kg to 20 mg/kg. Optimal doses for the therapeutic agents are the generally accepted efficacious doses, such as those described in the Physician Desk Reference, 56th Ed. (2002), Publisher Medical Economics, New Jersey. Optimal doses for agents not described in the Physician Desk Reference can be determined using a standard dose escalation study to identify the MTD (see, e.g., Richardson, et al. 2002, Blood, 100(9):3063-3067, the content of which is incorporated herein by reference).

In some embodiments, an anti-CS1 antibody is present in a pharmaceutical composition at a concentration, or in a weight/volume percentage, or in a weight amount, suitable for intravenous administration at a dosage rate at least about 0.5 mg/kg, at least about 0.75 mg/kg, at least about 1 mg/kg, at least about 2 mg/kg, at least about 2.5 mg/kg, at least about 3 mg/kg, at least about 4 mg/kg, at least about 5 mg/kg, at least about 6 mg/kg, at least about 7 mg/kg, at least about 8 mg/kg, at least about 9 mg/kg, at least about 10 mg/kg, at least about 11 mg/kg, at least about 12 mg/kg, at least about 13 mg/kg, at least about 14 mg/kg, at least about 15 mg/kg, at least about 16 mg/kg, at least about 17 mg/kg, at least about 18 mg/kg, at least about 19 mg/kg, and at least about 20 mg/kg.

### 6. EXAMPLES

### Example 1

### Lysis of MM Cells by HyLuc63

Gene expression was assessed using an Affymetrix GeneChip array. Protein expression was measured by flow cytometry, and immunohistochemistry (IHC), using HuLuc63, a novel humanized anti-CS1 mAb. HuLuc63-mediated lysis of myeloma cells via antibody dependent cellular cytotoxicity (ADCC) was measured by ⁵¹Cr-release.

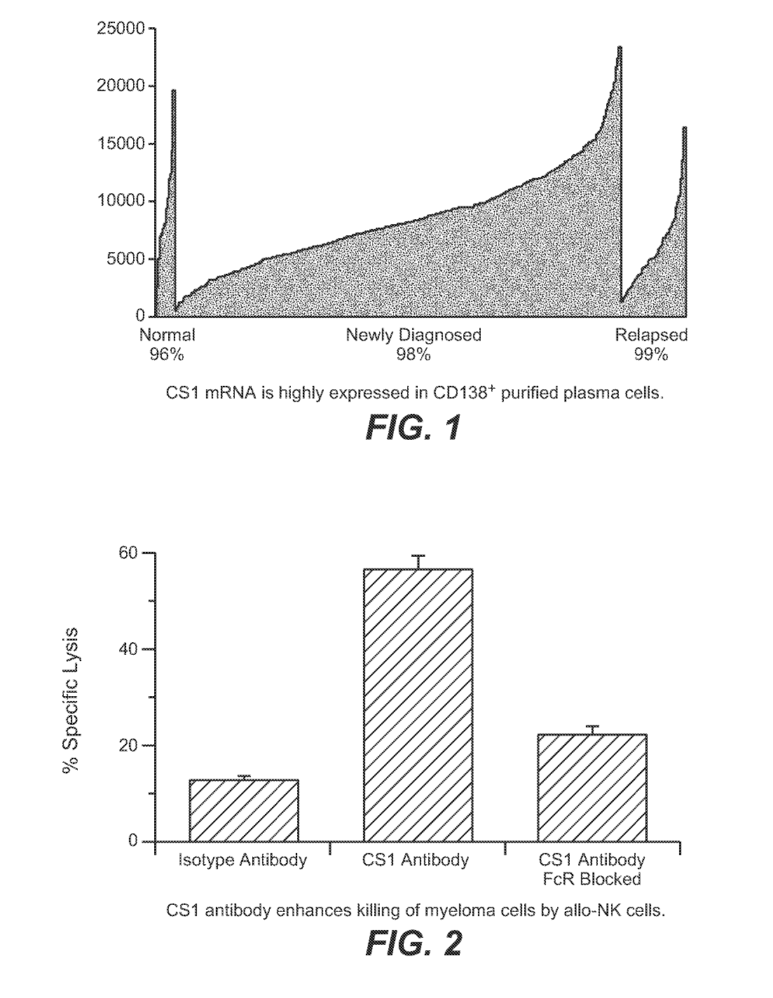

CS1 mRNA was detected in CD138+ purified plasma cells from >95% of healthy donors, newly diagnosed myeloma patients, and those with relapsed myeloma (FIG. 1). CS1 protein expression on primary myeloma cells was confirmed by flow cytometry, while IHC analysis of normal tissues revealed anti-CS1 staining primarily on CD138+ tissue plasma cells. Finally, we determined that HuLuc63 could induce killing of myeloma cells using purified allogeneic NK cells (FIG. 2). Blocking the Fc receptor greatly reduced this activity indicating an ADCC mechanism. Killing of myeloma targets was also observed in autologous systems suggesting that HuLuc63 can overcome KIR-mediated inhibition of autologous NK cells. In summary, we observed high mRNA and protein expression of CS1 in myeloma from early stage, late stage, and drug-treated patients, and showed enhanced lysis of myeloma when treated in vitro with HuLuc63. Our data support the potential clinical utility of CS1-targeted therapy.

### Example 2

### Haplo-Identical NK Cell Therapy Combined with HuLuc63 And Delayed Autograft

This therapy is intended for subjects who have relapsed myeloma or myeloma with disease progression. Typically, the therapy consists of five phases: Phase I: Induction chemotherapy and stem cell collection; Phase II: Conditioning regimen; Phase III, Collection of donor cells and administration of donor NK cells, Phase IV: Administration of Interleukin 2, and Phase V: Autologous transplant.

Phase I: Induction Chemotherapy and Stem Cell Collection

If the subjects do not have stored stem cells, stem cells can be collected during recovery from chemotherapy. DTPACE or other appropriate chemotherapeutic agents can be given to reduce the tumor burden prior to autotransplant. Following NK cell infusion or autotransplant, subjects can receive GM-CSF, until the bone marrow recovers and/or to assist peripheral blood stem cell collection. Other growth factors, such as G-CSF and EPO and/or antibiotics can be administered to the subject at the discretion of the investigator.

Phase II: Conditioning Regimen

A dose of 25 mg/m² fludarabine will be infused 5, 4, 3, and 2 days prior to infusion of donor NK cells into the subject. Fludarabine is typically administered by intravenous infusion over 30 minutes in 100 ml of normal saline (0.9%).

Dexamethasone, 40 mg ever day, will be given 5, 4, 3, and 2 days prior to infusion of donor NK cells into the subject. If required, an anti-emetic, such as Granisetron, can be administered at 2 mg orally, or 1 mg intravenous 5, 4, 3, 2, and 1 day prior to the infusion of donor NK cells into the subject. Melphalan will be given as a single dose of 140 mg/m², 1 day prior to infusion of donor NK cells into the subject.

Phase III: Collection of Donor Cells and Administration of Donor NK Cells and HuLuc63

Donors will not be given any colony stimulating factors prior to the collection of donor cells. A large volume leukapheresis to collect donor cells will be performed on days 0 and 2. The target number of NK cells to be infused is 0.5×10⁶−4×10⁷ NK cells/kg. The NK cells will be infused on days 0 and 2.

For infusion, the NK cells will be suspended in normal saline and 5% human albumin and transfused over approximately 8 hours by gravity. The recipient (i.e., subject) will receive standard monitoring for receiving cell products from a donor.

Subjects will receive an intravenous infusion of HuLuc63 prior to the NK donor cell infusions on days 0 and 2. Depending on the need of the subject and at the discretion of the investigator, HuLuc63 can be administered at dose levels ranging from 0.5 mg/kg to 20 mg/kg.

Phase IV: Administration of Interleukin 2

Interleukin 2 at 3×10⁶ U will be given subcutaneously beginning on the first day following completion of NK infusion to day 13. Subjects will be prehydrated with normal saline and given prophylactic dopamine infusion for renal protection. The dose of dopamine will not exceed 5 mcg/kg. Subjects will be pre-medicated as per the existing standard of care. If required, the dose of interleukin 2 can be adjusted and/or anti-histamines administered if redness at the site of injection occurs, or if systemic symptoms, e.g., fever or itch, are observed.

Phase V: Autologous Transplant

Peripheral blood stem cell infusion will be given intravenously on or after day 14. The subject will be premedicated according to standard practice. In general, approximately 3-6 10⁶/kg CD34⁺ cells will be infused with the autotransplant.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

What is claimed is:

1. A method of treating multiple myeloma in a subject, the method comprising administering an effective amount of allogeneic effector cells in combination with an effective amount of HuLuc63.

2. The method according to claim 1, wherein said allogeneic effector cells are lymphoid or myeloid cells or a combination thereof.

3. The method according to claim 1, wherein said allogeneic effector cells are lymphoid cells selected from the group consisting of T cells, NK cells, B cells and/or a combination thereof.

4. The method according to claim 3, wherein said allogeneic effector cells are alloreactive NK cells.

5. The method according to claim 1, wherein said allogeneic effector cells are derived from a donor, matched or mismatched in HLA type to the host.

6. The method according to claim 1, further comprising administration of a conditioning regimen comprising fludarabine, dexamethasone and melphalan.

7. The method according to claim 6, wherein the conditioning regimen is administered prior to the administration of the allogeneic effector cells.

8. The method according to claim 1, wherein HuLuc63 is administered prior to the administration of the allogeneic effector cells.

9. The method according to claim 8, in which HuLuc63 is administered intravenously at a dosage from approximately 0.5 mg/kg to approximately 20 mg/kg.

10. The method according to claim 1, wherein prior to the administration of the allogeneic effector cells and HuLuc63, the subject has undergone stem cell transplantation.

11. The method according to claim 10, wherein the stem cell transplantation is autologous stem cell transplantation.

12. The method according to claim 11, further comprising treating the subject with a maintenance regimen comprising the administration of HuLuc63.

13. The method according to claim 12, further comprising the administration of at one or more therapeutic agents.

14. The method according to claim 13, wherein at least one of the therapeutic agents is a targeted agent, a conventional chemotherapeutic agent, a hormonal therapy agent or a supportive care agent.

15. The method according to claim 1, wherein said subject is human.

16. The method according to claim 1, wherein said administration elicits a complete response.

17. The method according to claim 1, wherein said administration elicits a very good partial response.

18. The method according to claim 1, wherein said administration elicits a partial response.

19. The method according to claim 1, wherein said administration elicits a minimal response.

20. The pharmaceutical composition according to claim 1, comprising a first pharmaceutical composition comprising a therapeutically effective amount of allogeneic effector cells and a second pharmaceutical composition comprising HuLuc63.

21. The pharmaceutical composition according to claim 20, wherein said allogeneic effector cells are lymphoid or myeloid cells or a combination thereof.

22. The pharmaceutical composition according to claim 21, wherein said allogeneic effector cells are lymphoid cells selected from the group consisting of T cells, NK cells, B cells and/or a combination thereof.

23. The pharmaceutical composition according to claim 22, wherein said allogeneic effector cells are alloreactive NK cells.

24. The pharmaceutical composition according to claim 20, wherein HuLuc63 is administered at a dosage from approximately 0.5 mg/kg to approximately 20 mg/kg.

25. The pharmaceutical composition according to claim 20, wherein administration of said pharmaceutical composition elicits a complete response.

26. The pharmaceutical composition according to claim 20, wherein administration of said pharmaceutical composition elicits a very good partial response.

27. The pharmaceutical composition according to claim 20, wherein administration of said pharmaceutical composition elicits a partial response.

28. The pharmaceutical composition according to claim 20, wherein administration of said pharmaceutical composition elicits a minimal response.


SEQUENCE LISTING (6 sequences)

SEQ ID NO: 1 - protein, 335 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 2 - protein, 227 aa, Artificial
  hu50/mu50 amino acids 1-151 of human CS1 fused to amino acids 149-224 of mouse CS1
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTIDRQSNKNGTCVINLTCSTDQDGENVTYSWKAVGQGDNQFHDGATLSIAWRSGEKDQALTCMARNPVSNSFSTPVFPQKLCEDAATDLTSLRG

SEQ ID NO: 3 - protein, 227 aa, Artificial
  mu25/hu75 amino acids 1-67 of mouse CS1 fused to amino acids 68-227 of human CS1
MARFSTYIIFTSVLCQLTVTAASGTLKKVAGALDGSVTFTLNITEIKVDYVVWTFNTFFLAMVKKDGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMV

SEQ ID NO: 4 - protein, 224 aa, Artificial
  mu75/hu25 amino acids 1-166 of mouse CS1 fused to amino acids 170-227 of human CS1
MARFSTYIIFTSVLCQLTVTAASGTLKKVAGALDGSVTFTLNITEIKVDYVVWTFNTFFLAMVKKDGVTSQSSNKERIVFPDGLYSMKLSQLKKNDSGAYRAEIYSTSSQASLIQEYVLHVYKHLSRPKVTIDRQSNKNGTCVINLTCSTDQDGENVTYSWKAVGQAANESHNGSILPISWRWGESDMTFICVARNPVSRNFSSPILARKLCEGAADDPDSSMV

SEQ ID NO: 5 - protein, 119 aa, Homo saiens
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSS

SEQ ID NO: 6 - protein, 107 aa, Homo sapiens
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIK